(19) United States
(12) Patent Application Publication
(10) Pub. No.: US 2025/0099730 A1
(43) Pub. Date: Mar. 27, 2025

(54) Port Identification Insert and Methods

(71) Applicant: Bard Peripheral Vascular, Inc., Franklin Lakes, NJ (US)

(72) Inventor: Bryon Ray Densley, Rochester, MN (US)

(21) Appl. No.: 18/730,620

(22) PCT Filed: Jan. 21, 2022
(86) PCT No.: PCT/US22/13374

§ 371 Date: Jul. 19, 2024

(51) Int. Cl. A61M 39/02; A61M 39/00

(52) U.S. Cl. CPC A61M 39/0208; A61M 2039/0036; A61M 2039/0229; A61M 2039/0238

(57) ABSTRACT

Embodiments disclosed herein are directed to a subcutaneous access port including an identification insert, or marker, visible under medical imaging. An outer perimeter of the marker can define an alphanumeric symbol to indicate a property of the port, further the marker can indicate a location or orientation of the port. The marker can be received within a recess, a shape of the recess is configured to receive the marker in a first orientation. In an embodiment, the shape of the marker and recess is configured to prevent the marker from engaging the recess in a second orientation. This can prevent the port from being assembled if the marker is coupled with the port incorrectly. The port can further include a cover plate to secure the marker within the recess. A bottom surface of the cover plate can align with the bottom surface of the port when engaged therewith.

### SUMMARY

Briefly summarized, embodiments disclosed herein are directed to an identification insert or marker for a subcutaneous access port and associated methods of manufacture. A subcutaneous vascular access device, or “port,” can include an identification marker assembled with the device and configured to be visible under one or more medical imaging techniques (e.g. x-ray, fluoroscopy, ultrasound, MRI, CT, etc.) when the port is placed subcutaneously. The marker can indicate to a user a property of the port and can indicate a location and orientation of the port to facilitate access thereto. Various identification inserts or markers have been developed that align with a septum of the device to indicate a target location. Other port designs include identification inserts or markers positioned in a nose portion, disposed opposite a stem portion, so as not to be obscured when accessing the septum. Some identification markers, termed “positive” markers include an insert having an outer perimeter that defines a symbol, e.g. an alphanumeric symbol, icon, or the like, which can be identified under medical imaging. By contrast, “negative” identification markers, can include a plate or disc including an aperture extending therethrough. The perimeter of the aperture can define a shape or symbol (e.g. “CT”) that can be identified under medical imaging.

However, some of these identification markers can be assembled with the port incorrectly. As such, when imaging the subcutaneous port under medical imaging, the identification marker appears to indicate that the port has flipped or is otherwise incorrectly positioned, when the port is instead positioned correctly. This can lead to unnecessary disruptions and complications to the patient to correct the problem. Further, the outer dimensions of the “negative” identification markers are required to be larger than a minimum symbol size that is identifiable under medical imaging. Disclosed herein are devices and methods that address the foregoing.

Disclosed herein is a subcutaneous access port including, a base defining a reservoir and optionally having a nose portion (further optionally extending longitudinally, i.e. in a longitudinal direction), a stem in fluid communication with the reservoir and extending from the base (optionally in a longitudinal direction and/or opposite the nose portion), a first recess (disposed) in a bottom surface of the base, a radiopaque marker, an outer perimeter thereof defining a marker symbol and matching a shape of the first recess such that the radiopaque marker is configured to fit within the first recess (in a first orientation), and a cover plate disposed over and/or covering the first recess and coupled to the base to secure the radiopaque marker within the first recess.

In some embodiments, in a transverse direction perpendicular to the longitudinal direction, the radiopaque marker defines a transverse height, the first recess defines a transverse depth that is equal to or greater than the transverse height of the radiopaque marker.

In some embodiments, the first recess is disposed on a bottom surface of the nose portion of the base.

In some embodiments, the cover plate extends under a portion of the base disposed transversely below the reservoir and/or the cover plate is at least partially received within the base and is coupled to the base using one of adhesive, bonding, or welding.

In some embodiments, the base further includes a lip extending circumferentially about the first recess and configured to engage the cover plate in one of an interference fit, press fit, or snap fit engagement.

In some embodiments, the lip extends transversely downwards from the bottom surface of the base to secure the cover plate over the first recess.

In some embodiments, the lip extends from the bottom surface of the base transversely upwards into the base to define a second recess, the first recess disposed within the second recess, and the cover plate configured to fit within second recess.

In some embodiments, a shape of a perimeter of the lip matches a shape of a perimeter of the cover plate, and, in a transverse direction perpendicular to the longitudinal direction, a transverse depth of the lip is equal to or larger than a transverse height of the cover plate.

In some embodiments, the cover plate includes a cover plate symbol disposed thereon, the cover plate symbol optionally matching that of the marker symbol.

In some embodiments, the marker symbol is structured to indicate a property of the port.

In some embodiments, the marker symbol includes one or more of the letters “C,” “T,” and/or a conjoined symbol indicating the letters “CT”.

In some embodiments, the reservoir is disposed between the stem and the radiopaque marker along the longitudinal axis/direction.

In some embodiments, the port further includes a septum at least partially positioned over the reservoir and a cap configured to engage the base and secure the septum to the base.

Also disclosed is a method of manufacturing a subcutaneous access port, including providing a base defining a reservoir and optionally including a nose portion (further optionally extending longitudinally, i.e. in a longitudinal direction), the base including a stem extending therefrom (optionally in a longitudinal direction and/or opposite the nose portion), providing, optionally forming, a radiopaque marker, the perimeter of the marker defining a marker symbol, placing the radiopaque marker within a first recess (disposed) in a bottom surface of the base, optionally of the nose portion, in a first orientation, a shape of the recess configured to prevent the radiopaque marker from engaging (being received in) the recess in a second orientation different from the first orientation, and securing the radiopaque marker within the recess.

In some embodiments, in a transverse direction perpendicular to the longitudinal direction, the radiopaque marker defines a first transverse height and the first recess defines a first transverse depth equal to the first transverse height of the radiopaque marker.

In some embodiments, securing the radiopaque marker within the first recess includes one or more of a press-fit, interference fit, snap-fit engagement, adhesive, bonding, and welding.

In some embodiments, securing the radiopaque marker within the first recess includes coupling a cover plate to the base using one or more of a press-fit, interference fit, snap-fit engagement, adhesive, bonding, and welding, the cover plate extending over/covering the first recess.

In some embodiments, a portion of the cover plate extends underneath a portion of the base, below the reservoir, and/or the cover plate is at least partially received within the base.

In some embodiments, the first recess is disposed within a second recess disposed in the bottom surface of the base, the cover plate configured to fit within the second recess.

In some embodiments, the cover plate includes a cover plate symbol disposed thereon, the cover plate symbol optionally matching that of the marker symbol.

In some embodiments, the marker symbol includes the letters “C,” “T,” or a conjoined symbol indicating the letters “CT” and indicates a property of the port.

In some embodiments, a method comprises manufacturing an access port, as described herein.

### DRAWINGS

A more particular description of the present disclosure will be rendered by reference to specific embodiments thereof that are illustrated in the appended drawings. It is appreciated that these drawings depict only typical embodiments of the invention and are therefore not to be considered limiting of its scope. Example embodiments of the invention will be described and explained with additional specificity and detail through the use of the accompanying drawings in which:

FIG. 1A shows a perspective top-side view of a port including an identification marker, in accordance with embodiments disclosed herein.

FIG. 1B shows a perspective under-side view of a port including an identification marker, in accordance with embodiments disclosed herein.

FIG. 2 shows an exploded under-side view of a port including an identification marker, in accordance with embodiments disclosed herein.

FIG. 3 shows a lateral cross-section view of a nose portion of a port including an identification marker, in accordance with embodiments disclosed herein.

FIG. 4A shows an exploded under-side view of a port including an identification marker, in accordance with embodiments disclosed herein.

FIG. 4B shows a cross-section view of a portion of the port of FIG. 4A, in accordance with embodiments disclosed herein.

FIG. 5A shows an under-side view of an identification marker, in accordance with embodiments disclosed herein.

FIG. 5B shows a top-side view of an identification marker, in accordance with embodiments disclosed herein.

FIG. 5C shows a perspective view of an identification marker, in accordance with embodiments disclosed herein.

FIGS. 5D-5G show various side views of the identification marker of FIG. 5C, in accordance with embodiments disclosed herein.

### DESCRIPTION

Before some particular embodiments are disclosed in greater detail, it should be understood that the particular embodiments disclosed herein do not limit the scope of the concepts provided herein. It should also be understood that a particular embodiment disclosed herein can have features that can be readily separated from the particular embodiment and optionally combined with or substituted for features of any of a number of other embodiments disclosed herein.

Regarding terms used herein, it should also be understood the terms are for the purpose of describing some particular embodiments, and the terms do not limit the scope of the concepts provided herein. Ordinal numbers (e.g., first, second, third, etc.) are generally used to distinguish or identify different features or steps in a group of features or steps, and do not supply a serial or numerical limitation. For example, “first,” “second,” and “third” features or steps need not necessarily appear in that order, and the particular embodiments including such features or steps need not necessarily be limited to the three features or steps. Labels such as “left,” “right,” “top,” “bottom,” “front,” “back,” and the like are used for convenience and are not intended to imply, for example, any particular fixed location, orientation, or direction. Instead, such labels are used to reflect, for example, relative location, orientation, or directions. Singular forms of “a,” “an,” and “the” include plural references unless the context clearly dictates otherwise.

In the following description, the terms “or” and “and/or” as used herein are to be interpreted as inclusive or meaning any one or any combination. As an example, “A, B or C” or “A, B and/or C” mean “any of the following, A, B, C, A and B, A and C, B and C, A, B and C.” An exception to this definition will occur only when a combination of elements, components, functions, steps or acts are in some way inherently mutually exclusive.

With respect to “proximal,” a “proximal portion” or a “proximal end portion” of, for example, a catheter disclosed herein includes a portion of the catheter intended to be near a clinician when the catheter is used on a patient. Likewise, a “proximal length” of, for example, the catheter includes a length of the catheter intended to be near the clinician when the catheter is used on the patient. A “proximal end” of, for example, the catheter includes an end of the catheter intended to be near the clinician when the catheter is used on the patient. The proximal portion, the proximal end portion, or the proximal length of the catheter can include the proximal end of the catheter; however, the proximal portion, the proximal end portion, or the proximal length of the catheter need not include the proximal end of the catheter. That is, unless context suggests otherwise, the proximal portion, the proximal end portion, or the proximal length of the catheter is not a terminal portion or terminal length of the catheter.

With respect to “distal,” a “distal portion” or a “distal end portion” of, for example, a catheter disclosed herein includes a portion of the catheter intended to be near or in a patient when the catheter is used on the patient. Likewise, a “distal length” of, for example, the catheter includes a length of the catheter intended to be near or in the patient when the catheter is used on the patient. A “distal end” of, for example, the catheter includes an end of the catheter intended to be near or in the patient when the catheter is used on the patient. The distal portion, the distal end portion, or the distal length of the catheter can include the distal end of the catheter; however, the distal portion, the distal end portion, or the distal length of the catheter need not include the distal end of the catheter. That is, unless context suggests otherwise, the distal portion, the distal end portion, or the distal length of the catheter is not a terminal portion or terminal length of the catheter.

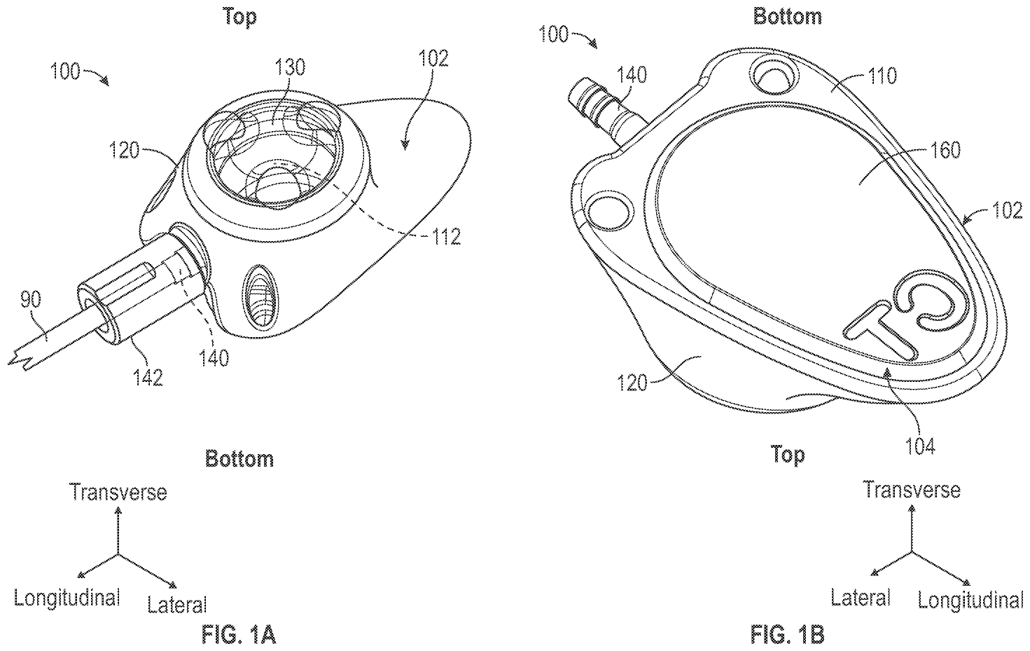

To assist in the description of embodiments described herein, as shown in FIGS. 1A-1B, a longitudinal axis extends substantially parallel to an axial length of the port stem. A lateral axis extends normal to the longitudinal axis, and a transverse axis extends normal to both the longitudinal and lateral axes. A horizontal plane is defined by the longitudinal and lateral axes. A vertical plane extends perpendicular to the horizontal axis.

Unless defined otherwise, all technical and scientific terms used herein have the same meaning as commonly understood by those of ordinary skill in the art.

FIGS. 1A-1B show an exemplary subcutaneous access device, or “port” 100. FIG. 1A shows a top-side perspective view of the port 100. FIG. 1B shows an under-side perspective view of the port 100. The port 100 can generally include a base 110 that defines a reservoir 112, and includes a stem 140 extending therefrom along a longitudinal axis. The stem 140 can define a stem lumen that is in fluid communication with the reservoir 112. A needle penetrable septum 130 can be disposed over the reservoir 112 and can be secured in place by a cap 120. The septum 130 can be formed of a silicone rubber or similar suitable material. In use, when the port 100 is placed subcutaneously, a top side of the port 100 is disposed proximate a skin surface. A user can penetrate the skin using an access needle (not shown) and can penetrate a septum 130 to access the reservoir 112.

The cap 120 can engage the base 110 in an ultrasonically welded, press-fit, interference fit, or snap fit engagement. In an embodiment, the cap 120 can be secured to the base 110 using adhesive, bonding, welding, ultrasonic welding, or similar suitable means. In an embodiment, the cap 120 can be over-molded on to the base 110. In an embodiment, the cap 120 and the base 110 can be formed as a single monolithic piece formed of the same material.

In an embodiment, the base 110 can be formed of a substantially rigid or resilient material including, but not limited to, a plastic, polymer, thermoplastic, metal, alloy, composite, combinations thereof, or the like. In an embodiment, the cap 120 can be formed of a softer material relative to the base 110 including, but not limited to, a plastic, polymer, thermoplastic, elastomer, rubber, silicone rubber, metal, alloy, composite, combinations thereof, or the like. In an embodiment, the base 110 and the cap 120 can be formed of the same material and can display the same mechanical properties. In an embodiment, a catheter 90 can engage the stem 140 in one of a press-fit or interference fit engagement. Optionally a cathlock 142 can engage one or both of the catheter 90 and the stem 140 to further secure the catheter 90 to the stem 140.

In an embodiment, the port 100 can include a nose portion 102 extending from the port 100 along a longitudinal axis and disposed opposite the stem 140 across a central transverse axis of the reservoir 112. The nose portion 102 can define a substantially wedge shaped profile extending from the port 100. In an embodiment, the nose portion 102 can be configured to facilitate subcutaneous placement thereof. For example, as the port 100 is urged subcutaneously, the nose portion 102 can facilitate separation of subcutaneous tissues to form a tissue pocket.

In an embodiment, the nose portion 102 can include a symbol 104, e.g. an alphanumeric symbol, shape, icon, or the like, configured to indicate a property of the port 100. For example, as shown in FIG. 1B, a bottom surface of the base 110 or a cover plate 160 can include a symbol 104 such as the letters “CT” which is designed to indicate a property of the port 100 such as the port 100 is configured for high pressure infusion, or the like. These and other properties of the port 100 including make, model, part number, serial number, website addresses, bar codes, QR codes, combinations thereof, or the like are contemplated to fall within the scope of the present invention. The symbol 104 can be printed, embossed, or engraved onto or into the base 110 or the cover plate 160 and can be directly observable by a user to visually indicate one or more properties of the port 100. As will be appreciated the port 100 can include one or more symbols 104 disposed on one or more surfaces of the port 100, without limitation.

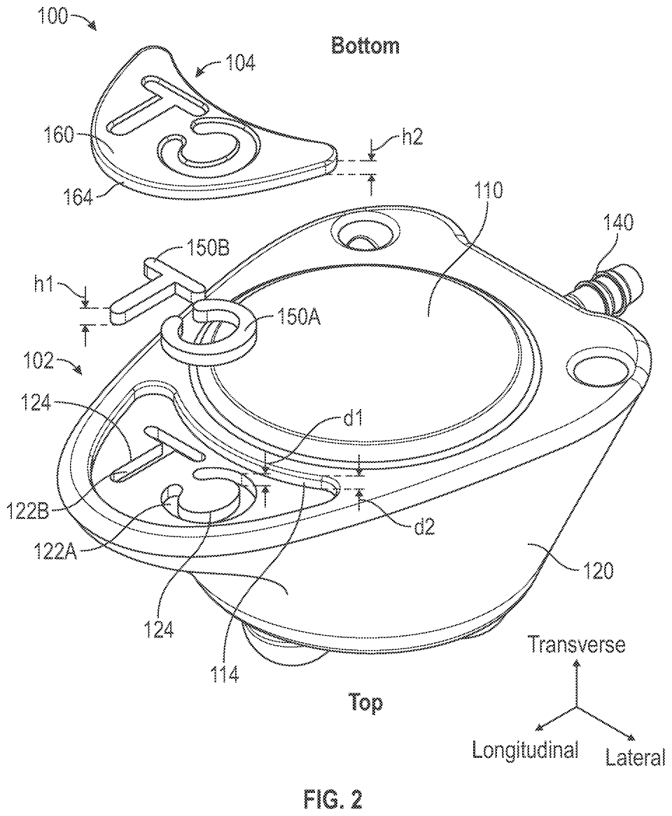

FIG. 2 shows an exploded view of a port 100 generally including a port base 110, an identification insert or an identification marker (“marker”) 150, and a cover plate 160. In an embodiment, a bottom surface of the base 110 can include a recess 122 disposed in the nose portion 102 of the port 100 and extending over a horizontal plane. The recess 122 can also extend transversely to define a first depth (d1). In an embodiment, a perimeter 124 of the recess 122 can define curvilinear or polygonal shape extending over the horizontal plane. As shown, the perimeter 124 can define a shape that matches the shape of an outer perimeter of the marker 150. It will be appreciated, however, that other curvilinear or polygonal shapes are also contemplated.

In an embodiment, the shape of the perimeter 124 of the recess 122 can be equal to or larger than a perimeter 154 of the marker 150. As such, the marker 150 can slidably engage the recess 122. In an embodiment, the marker 150 can be secured within the recess 122 using adhesive, bonding, welding, or the like. In an embodiment, the shape of the perimeter 124 of the recess 122 can be equal to or slightly smaller than a perimeter 154 of the marker 150. As such, the marker 150 can engage the recess 122 in one of an interference fit, press fit, or snap fit engagement. In an embodiment, the depth (d1) of the recess 122 can be equal to or larger than a transverse height (h1) of the marker 150. As such, the marker 150 can be configured to fit within the recess 122. In an embodiment, a transverse height (h1) of the marker 150 can be between 0.20 and 0.60 inches. However, greater or lesser dimensions are also contemplated to fall within the scope of the present invention.

In an embodiment, the port 100 can include one or more recesses 122, each configured to receive a marker 150. For example, as shown in FIG. 2, a first recess 122A can define a “C” shape configured to receive a first marker 150A defining a “C” shape. Further, a second recess 122B can define a “T” shape configured to receive a second marker 150B defining a “T” shape. However, it will be appreciated that other shaped recesses 122 and markers 150 including different shapes, alphanumeric symbols, or the like, are also contemplated. In an embodiment, the alphanumeric shape (i.e. the “C” shape or the “T” shape) can be equivalent to between a 10 pt. and 16 pt. size font. However, greater or lesser font sizes are also contemplated.

In an embodiment, the marker 150 can be formed of a radiopaque material configured to be visible when imaged by one or more medical imaging techniques. Exemplary medical imaging techniques can include, but not limited to, x-ray, fluoroscopy, ultrasound, magnetic resonance imaging (MRI), computed tomography (CT), positron emission tomography (PET), combinations thereof, or the like.

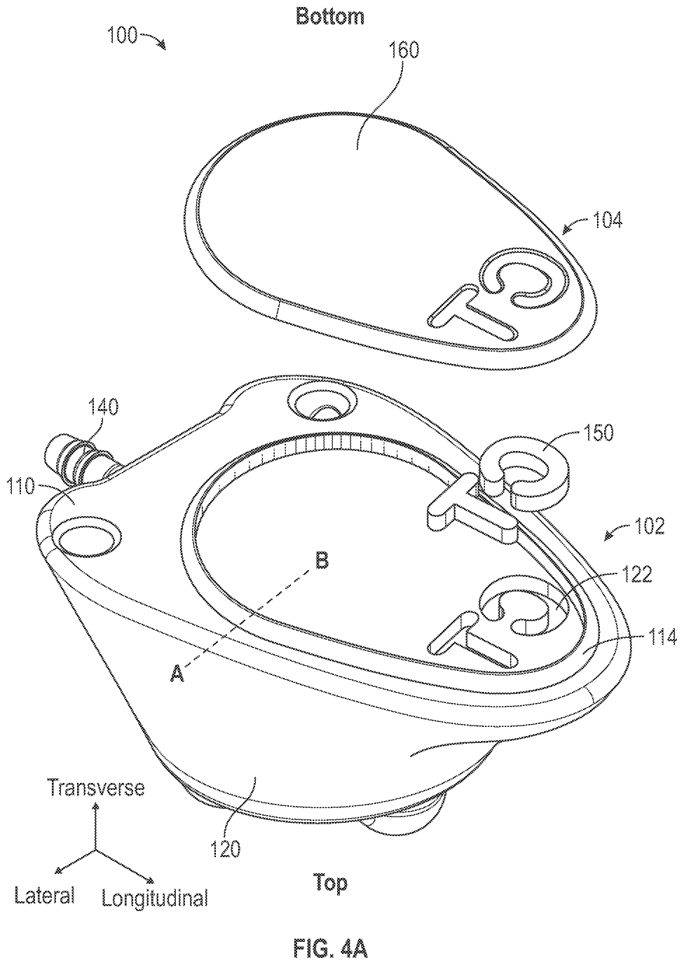

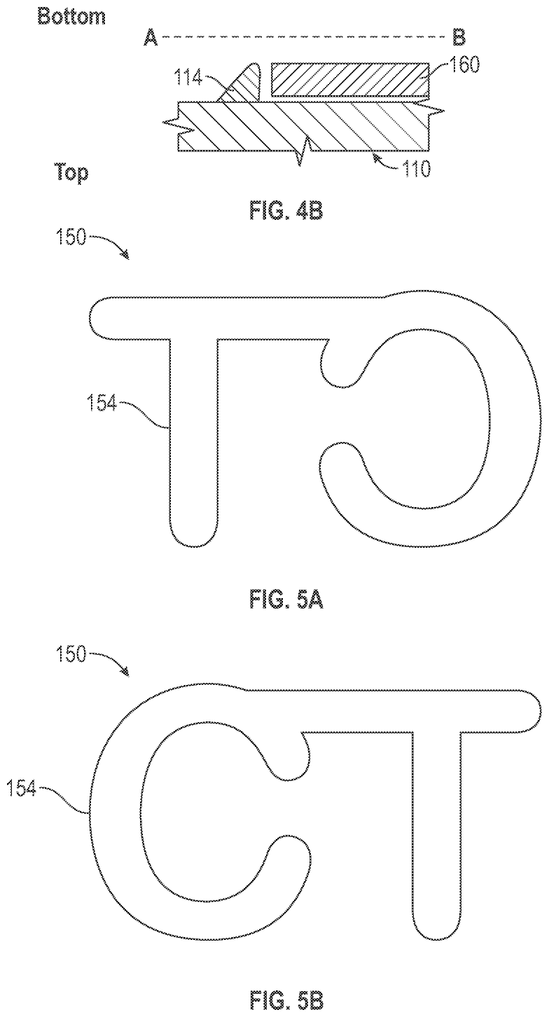

In an embodiment, the port 100 can further include a cover plate 160 configured to be disposed over the recess 122 and secure the marker 150 within the recess 122. In an embodiment, the cover plate 160 can be secured to the base 110 using adhesive, bonding, welding, or similar suitable means. In an embodiment, the base 110 can include a lip 114 extending circumferentially about at least a portion of the recess 122. A perimeter 164 of the cover plate 160 can engage the lip 114 in one of an interference fit, press-fit, or snap fit engagement to secure the cover plate 160 to the base 110. In an embodiment, as shown in FIGS. 4A-4B, the lip 114 can extend transversely downwards from a bottom surface of the base 110 so as to secure the cover plate 160 over the recess 122.

In an embodiment, as shown in FIG. 2, the lip 114 can extend transversely upwards into a bottom surface of the base 110 so as to secure the cover plate 160 over the recess 122. Worded differently, the lip 114 extending upwards into the base 110 can define a second recess, within which the first recess 122 is disposed. The first recess 122 is configured to receive the marker 150 and the second recess is configured to receive the cover plate 160. In an embodiment, the lip 114 can extend transversely by a depth (d2) which can be equal to or greater than a thickness (h2) of the cover plate 160. In an embodiment, the thickness (h2) of the cover plate 160 can be between 0.05 and 0.60 inches. However, greater or lesser dimensions are also contemplated to fall within the scope of the present invention. In an embodiment, a bottom surface of the cover plate 160 can align with a bottom surface of the base 110 when the cover plate 160 is engaged with the second recess, to provide a continuous bottom surface of the port 110.

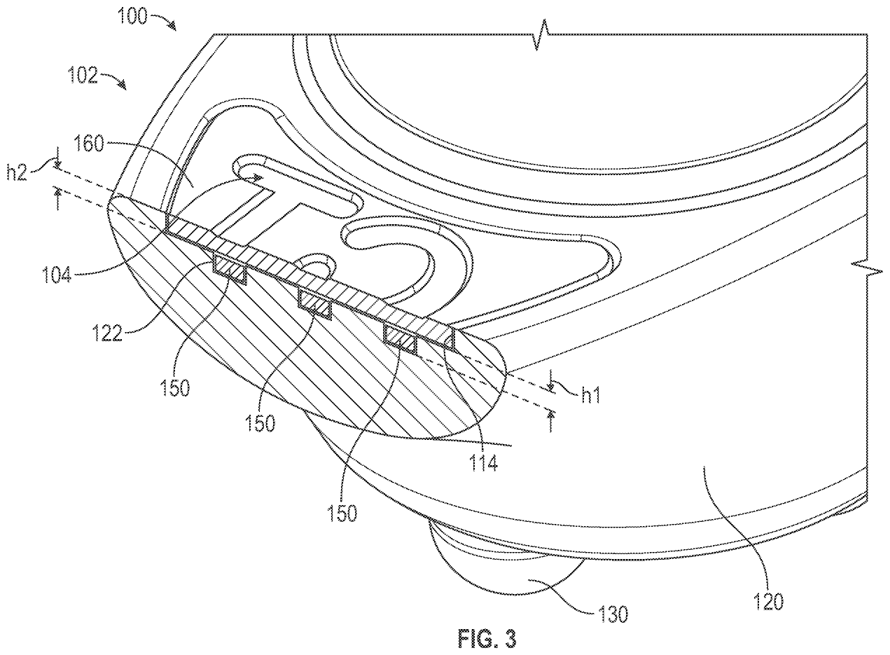

FIG. 3 shows a cross-section view of a nose portion 102 of the port 100 including the marker(s) 150 disposed within the recess(es) 122 and a cover plate 160 disposed thereover. FIG. 3 shows the lip 114 extending transversely upwards into the base 110. As such, the cover plate 160 is received within a second recess defined by the lip 114 such that the bottom surface of the cover plate 160 aligns with a bottom surface of the base 110.

In an embodiment, as shown in FIG. 4A, the recess 122 can extend transversely upwards into the base 110 from a bottom surface of the base 110. The cover plate 160 can extend over the recess 122 and extend over at least a portion of the bottom surface of base 110, transversely below reservoir 112. The cover plate 160 can be attached to the base 110 using adhesive, bonding, welding, or the like, as described herein. Advantageously, the cover plate 160 can both secure the marker 150 within the recess 122 and provide increased reinforcement to the base 110 below the reservoir 112 to mitigate the access needle from penetrating base 110 when accessing the reservoir 112.

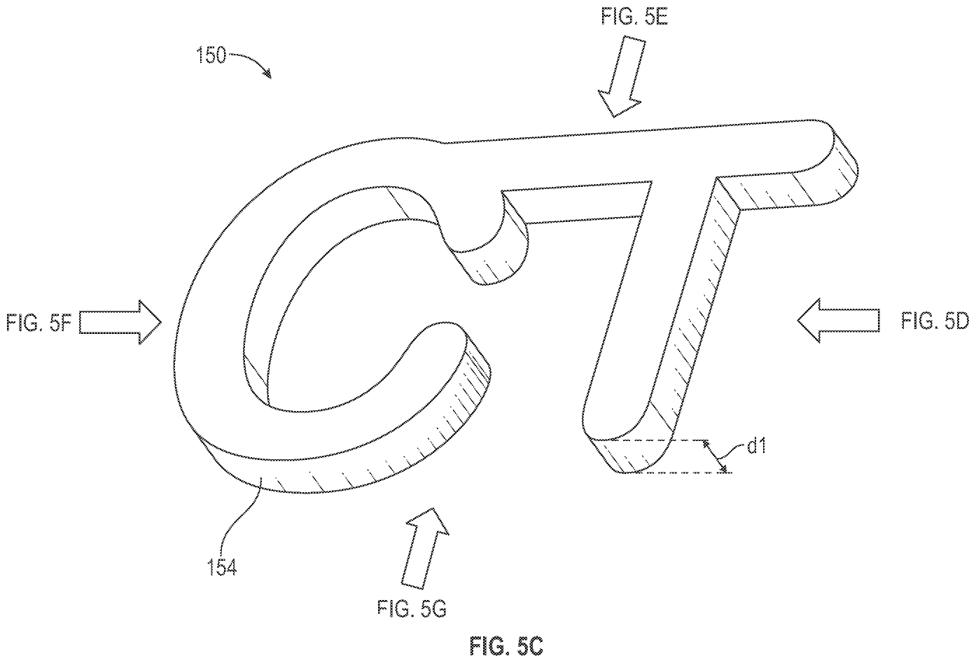

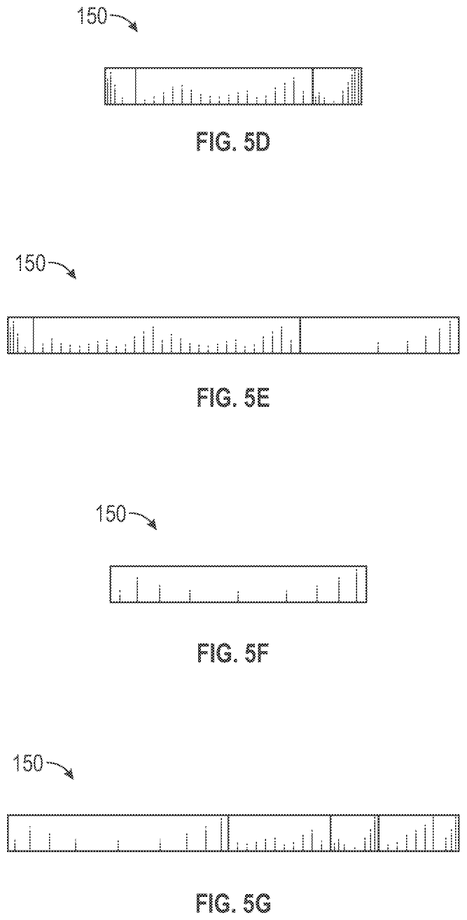

FIGS. 5A-5G show various views of an embodiment of a marker 150. FIG. 5A shows an under side view, FIG. 5B shows a top side view. FIG. 5C shows a perspective view of the marker 150. FIGS. 5D-5G show side views of the marker 150 as indicated in FIG. 5C. In an embodiment, a perimeter 154 of the marker 150 can define a shape that resembles two or more alphanumeric symbols joined together such that a single monolithic marker 150 can define two or more alphanumeric symbols identifiable when imaged under medical imaging. In an embodiment, a shape of the perimeter 124 of the recess 122 can match the shape of the perimeter 154 of the marker 150 such that the marker 150 can be received within the recess 122. Further, the recess 122 and the marker 150 can be configured such that the marker 150 can be received within the recess 122 in a first orientation in Cartesian space but cannot be received within the recess 122 in a second orientation, different from the first orientation, in Cartesian space. Advantageously, the marker 150 can only be assembled with the port 100 in the first orientation and cannot be assembled with the port in a second orientation, i.e. incorrectly. As such, when imaging the port using medical imaging techniques the correct orientation of the port 100 is indicated.

As will be appreciated, the marker 150 formed of radiopaque material will contrast with the surrounding, non-radiopaque material. As such, the perimeter 154 of the marker 150 can define a symbol when imaged under medical imaging techniques, as described herein. The marker symbol can then indicate one or more of a properties of the port 100, and/or a location or an orientation of the port 100 when imaged subcutaneously. Where the perimeter 154 of the marker 150 can define the symbol, the marker 150 can be termed a “positive” image marker. This contrasts with “negative” markers that include an aperture, or “punch-out,” the shape of the punch-out defining the symbol. Advantageously, positive markers can define smaller sized symbols and can be included with smaller ports 100. Worded differently, a positive marker requires a smaller overall footprint of the marker 150 relative to a negative marker defining a symbol of the same size, and as such the positive marker 150 can be included with smaller devices, such as port 100.

In an embodiment, the recess 122 can be disposed in a portion of the cap 120. A portion of the base 110 can extend over at least a portion of the recess 122. As such, with the marker 150 disposed within the recess, the base 110 can secure the marker 150 within the recess 122 in the first orientation. In an embodiment, a portion of the base 110 can extend over the entire recess 122 to seal the marker 150 within the recess 122. The base 110 can be attached to the cap 120 using adhesive, bonding, welding or similar suitable means. As will be appreciated, if the marker 150 is placed within the recess 122 in a second orientation, different from the first orientation, the marker 150 will be mis-aligned with the recess 122 and cannot be fully received within the recess 122, preventing the base 110 from fully engaging the cap 120. Advantageously, the marker 150 is configured to prevent the port 100 from being assembled incorrectly.

In an embodiment, the symbol 104 disposed on the lower surface of the cover plate 160 or the base 110 can align with the positive symbol defined by the marker 150. As such the symbol 104 can provide a visual representation of the symbol defined by the marker 150 disposed within the recess 122. In an embodiment, one or more of the cover plate 160, base 110 and the cap 120 can be formed of an opaque material. As such, the marker 150 may not be directly observable when assembled within port 100.

In an embodiment, as shown in FIGS. 1-4, the symbol 104 can include alphanumeric symbols engraved on a bottom surface of the cover plate 160 or the base 110 in reverse so as to align with the symbol(s) defined by marker 150. As such, the symbol 104 can visually convey the orientation of the marker 150 within the recess 122 and a user can observe the symbol 104 to confirm the marker 150 and thereby port 100 is positioned correctly during subcutaneous placement of the port 100. In an embodiment, the base can include a first symbol configured to align with the marker 150 disposed within the recess 122. The base 110 can further include a second symbol (not shown), as described herein and can be configured to convey a second property of the port 100, for example a make, model, serial number, or the like, as described herein.

In an embodiment, one or more of the cover plate 160, the base 110 or the cap 120 can be formed of a transparent or translucent material to allow a user to directly observe the marker 150 disposed within the recess 122 when the port 100 is assembled. In an embodiment, one or more of the cover plate 160, the base 110 or the cap 120 can include a window formed of a transparent or translucent material and aligned with the recess 122 to allow a user to directly observe the marker 150 disposed within the recess 122 when the port 100 is assembled. Advantageously, one or more of the cover plate 160, the base 110 or the cap 120 can co-operate to provide a smooth outer profile and can contain the marker 150 therein to prevent any tissue ingrowth into the aperture 152 when the port 100 is placed subcutaneously.

While some particular embodiments have been disclosed herein, and while the particular embodiments have been disclosed in some detail, it is not the intention for the particular embodiments to limit the scope of the concepts provided herein. Additional adaptations and/or modifications can appear to those of ordinary skill in the art, and, in broader aspects, these adaptations and/or modifications are encompassed as well. Accordingly, departures may be made from the particular embodiments disclosed herein without departing from the scope of the concepts provided herein.

1. A subcutaneous access port, comprising:
a base defining a reservoir and having a nose portion extending longitudinally;
a stem in fluid communication with the reservoir and extending from the base opposite the nose portion;
a first recess disposed in a bottom surface of the base;
a radiopaque marker, an outer perimeter thereof defining a marker symbol and matching a shape of the first recess such that the radiopaque marker is configured to fit within the first recess in a first orientation; and
a cover plate disposed over the first recess and coupled to the base to secure the radiopaque marker within the first recess.

2. The subcutaneous access port according to claim 1, wherein the radiopaque marker defines a transverse height, the first recess defines a transverse depth that is equal to or greater than the transverse height of the radiopaque marker.

3. The subcutaneous access port according to claim 1, wherein the first recess is disposed on a bottom surface of the nose portion of the base.

4. The subcutaneous access port according to claim 1, wherein the cover plate extends under a portion of the base disposed transversely below the reservoir and is coupled to the base using one of adhesive, bonding, or welding.

5. The subcutaneous access port according to claim 1, wherein the base further includes a lip extending circumferentially about the first recess and configured to engage the cover plate in one of an interference fit, press fit, or snap fit engagement.

6. The subcutaneous access port according to claim 5, wherein the lip extends transversely downwards from the bottom surface of the base to secure the cover plate over the first recess.

7. The subcutaneous access port according to claim 5, wherein the lip extends from the bottom surface of the base transversely upwards into the base to define a second recess, the first recess disposed within the second recess, and the cover plate configured to fit within second recess.

8. The subcutaneous access port according to claim 5, wherein a shape of a perimeter of the lip matches a shape of a perimeter of the cover plate, and a transverse depth of the lip is equal to or larger than a transverse height of the cover plate.

9. The subcutaneous access port according to claim 1, wherein the cover plate includes a cover plate symbol disposed thereon, the cover plate symbol matching that of the marker symbol.

10. The subcutaneous access port according to claim 1, wherein the marker symbol indicates a property of the port.

11. The subcutaneous access port according to claim 1, wherein the marker symbol includes one or more of the letters “C,” “T,” or a conjoined symbol indicating the letters “CT”.

12. The subcutaneous access port according to claim 1, wherein the reservoir is disposed between the stem and the radiopaque marker along the longitudinal axis.

13. The subcutaneous access port according to claim 1, wherein the port further includes a septum positioned over the reservoir and a cap configured to engage the base and secure the septum to the base.

14. A method of manufacturing a subcutaneous access port, comprising:
providing a base defining a reservoir and including nose portion extending longitudinally, the base including a stem extending therefrom opposite the nose portion;
forming a radiopaque marker, the perimeter of the marker defining a marker symbol;
placing the radiopaque marker within a first recess disposed in a bottom surface of the nose portion in a first orientation, a shape of the recess configured to prevent the radiopaque marker from engaging the recess in a second orientation different from the first orientation; and
securing the radiopaque marker within the recess.

15. The method according to claim to 14, wherein the radiopaque marker defines a first transverse height and the first recess defines a first transverse depth equal to the first transverse height of the radiopaque marker.

16. The method according to claim 14, wherein securing the radiopaque marker within the first recess includes one or more of a press-fit, interference fit, snap-fit engagement, adhesive, bonding, and welding.

17. The method according to claim 14, wherein securing the radiopaque marker within the first recess includes coupling a cover plate to the base using one or more of a press-fit, interference fit, snap-fit engagement, adhesive, bonding, and welding, the cover plate extending over the first recess.

18. The method according to claim 17, wherein a portion of the cover plate extends underneath a portion of the base, below the reservoir.

19. The method according to claim 14, wherein the first recess is disposed within a second recess disposed in the bottom surface of the base, the cover plate configured to fit within the second recess.

20. The method according to claim 14, wherein the cover plate includes a cover plate symbol disposed thereon, the cover plate symbol matching that of the marker symbol.

21. The method according to claim 14, wherein the marker symbol includes the letters “C,” “T,” or a conjoined symbol indicating the letters “CT” and indicates a property of the port.
